(19) United States
(12) Patent Application Publication
(10) Pub. No.: US 2014/0358025 A1
(43) Pub. Date: Dec. 4, 2014

(54) System and apparatus for seizure detection from EEG signals

(71) Applicants: Keshab K. Parhi, Maple Grove, MN (US); Zisheng Zhang, St. Paul, MN (US)

(72) Inventors: Keshab K. Parhi, Maple Grove, MN (US); Zisheng Zhang, St. Paul, MN (US)

(21) Appl. No.: 13/986,720

(22) Filed: May 29, 2013

(51) Int. Cl. A61B 5/04; A61B 5/00; A61N 1/36; A61N 2/00

(52) U.S. Cl. CPC A61B 5/04017; A61N 2/006; A61B 5/04004; A61B 5/4839; A61N 1/36064
USPC 600/544

(57) ABSTRACT

The present invention relates to the design and implementation of a seizure detection system. In this invention, a reliable way to detect seizures is presented. The proposed invention filters an EEG signal by a Prediction Error Filter. The output of the prediction error filter is subjected to wavelet decomposition. Various features are then extracted from the wavelet coefficients. These features are input to a classifier to detect seizures. The proposed algorithm takes advantage of high sensitivity in detecting seizures and low complexity in implementation. The proposed scheme is general and is suitable for creating a trigger for therapy delivery in a closed-loop therapy system. The therapy could involve either delivery of an anti-epileptic drug or electrical or magnetic stimulation of the brain.

### CROSS-REFERENCE TO RELATED APPLICATIONS

This application claims the benefit of U.S. Provisional Application No. 61/689,201, filed on May 31, 2012, the entire content of which is incorporated herein by reference in its entirety.

### FIELD OF THE INVENTION

Certain embodiments of the invention relate to processing of Electroencephalogram (EEG) signals to detect seizures in epileptic patients. More specifically, certain embodiments of the invention relate to a method and an apparatus for detecting seizures by using a prediction error filter, wavelet decomposition of the error output, computing features from these coefficients, and a classifier.

### BACKGROUND OF THE INVENTION

Approximately 1% of the world's population suffers from epilepsy which is the second most common neurological disorder and is characterized by seizures. Reliable seizure detection is therefore important for not only improving the lives of epileptic patients, but also in assisting the epileptologists in marking seizures in the Electroencephalogram (EEG) recordings. An apparatus that can detect seizures can be used in a closed-loop therapy system to deliver an anti-epilepsy drug or other therapy as needed.

Therefore, there is a current need for designing an algorithm for a wearable or an implantable device that can reliably detect seizures with low computational complexity. In particular, the algorithm should require low power consumption and low hardware cost when implemented in an apparatus that can detect seizures.

### BRIEF SUMMARY OF THE INVENTION

Methods for designing a system architecture that is able to reliably detect seizures are provided. The invention is suited for low-power biomedical monitoring systems for detecting seizures. In one embodiment of the invention, such an apparatus can trigger delivery of anti-epileptic drugs or other therapy. In another embodiment of the invention, the system can be used to mark seizures in an unmarked EEG recordings.

The present invention proposes a new algorithm and a system architecture for seizure detection. In one embodiment, the algorithm can be applied to a single EEG channel. In another embodiment, the algorithm can be applied to a plurality of channels. This algorithm can be coded in a computer language and then be executed by any computing device. The system architecture can also be implemented using digital circuits in a wearable or implantable device.

The seizure detection method includes preprocessing of a single-channel EEG data collected from a subject's brain. The EEG recording could be a scalp recording or an intra-cranial recording. The preprocessing removes the mean of the EEG signal. A key aspect of this invention is the use of a prediction error filter to compute a whitened error signal from the demeaned EEG signal. The prediction error filter coefficients are computed as needed. In one embodiment, these coefficients can be fixed. In another embodiment, these could be computed using few minutes of recording. These coefficients can be used for computing the prediction error filter output. The coefficients can be recomputed after a period of time. In one example, the coefficients could be computed once an hour. In another embodiment, these could be computed once a day. Other computing intervals can be used in other embodiments. This process comprises the following steps: (1) dividing the data into overlapping or non-overlapping segments (2) applying an auto-regression analysis to the windowed signal, and (3) computing the whitened signal by passing the signal through the prediction error filter.

Features are then extracted from the error signal for classification of seizure. The error signal is subjected to wavelet decomposition and features are computed from the wavelet coefficients. These features are then used by a classifier to detect seizures as described below.

The final step is to identify the onset of a seizure using uni-variate or multi-variate classifiers based on the said features. The classifier processes the features and computes a decision variable that is thresholded to classify and detect seizures. In other embodiment, a post-processing step is applied to the decision variable of the classifier to reduce undesired noisy fluctuations. The output of the postprocessing step is then thresholded to classify and detect seizure. Such a post-processing could be carried out by a moving-average filter or a median filter or a Kalman filter.

### BRIEF DESCRIPTION OF THE DRAWINGS/FIGURES

The present invention is described with reference to the accompanying figures. The accompanying figures, which are incorporated herein, form part of the specification, illustrate the present invention, and together with the description further serve to explain the principles of the invention and to enable a person skilled in the relevant art to make and use the invention.

FIG. 1 illustrates the raw EEG signals in preictal, ictal and interictal time period.

FIG. 2 illustrates the block diagram of a basic seizure detection algorithm.

FIG. 3 illustrates the block diagram of an advanced seizure detection algorithm.

FIG. 4 illustrates the block diagram of the proposed seizure detection algorithm.

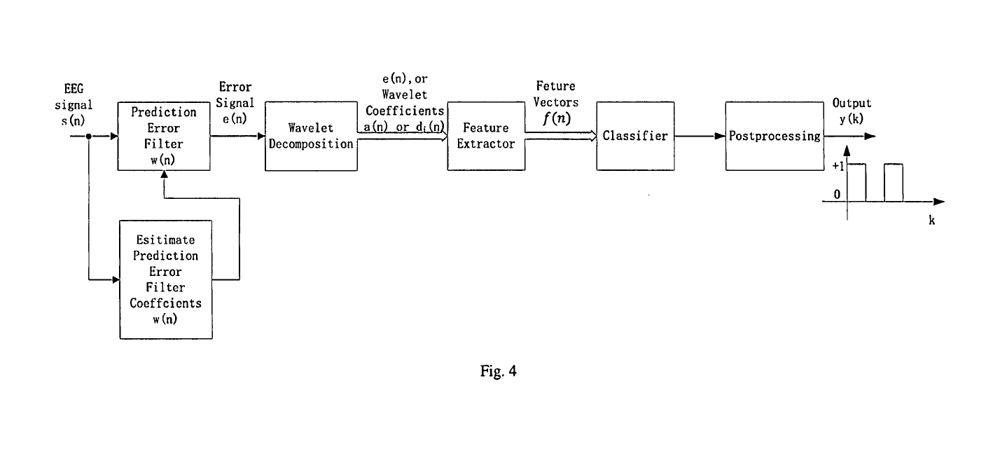

FIG. 5 illustrates the block diagram of a prediction error filter.

FIG. 6 illustrates the block diagram of a one-level wavelet decomposition with feature extraction.

FIG. 7 illustrates the block diagram of a 2-level wavelet decomposition with feature extraction.

FIG. 8 illustrates the block diagram of a 3-level wavelet decomposition with feature extraction.

FIG. 9 illustrates the block diagram of a feature extractor using either mean-squared or absolute values of the input coefficients.

FIG. 10 illustrates the block diagram of a feature extractor using the ratio of either mean-squared or absolute values of the two input coefficients.

FIG. 11 illustrates multiple features extracted using a 2-level wavelet decomposition and mean squared coefficients of (a) e(n), (b) a₂(n), (c) d₂(n) and (d) d₁(n).

FIG. 12 illustrates the block diagram of a multi-variate classifier.

FIG. 13 illustrates the block diagram of an ADABOOST using decision stumps as basic learners.

FIG. 14 illustrates an implementation of the ADABOOST classifier using serial processing.

FIG. 15 illustrates the block diagram of the proposed seizure detection algorithm with ADABOOST classifier.

### DETAILED DESCRIPTION OF THE INVENTION

Seizure detection has been of great interest in past decades. Various algorithms have been proposed to reliably detect the seizures with reduced computational complexity.

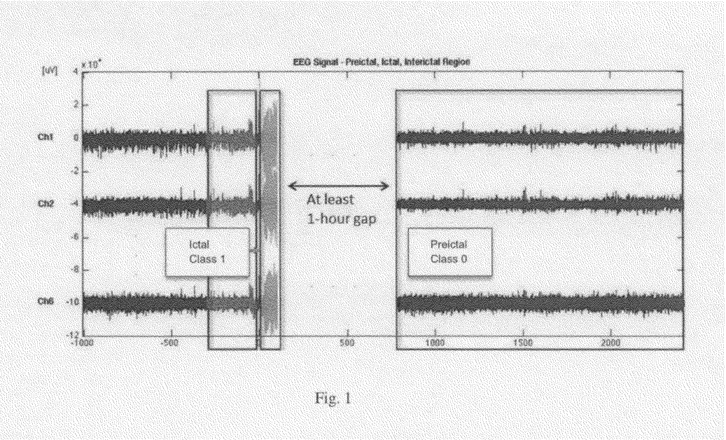

A seizure detection problem can be viewed as a binary classification problem, where one class consists of ictal signals corresponding to an occurrence of the seizure, and the other class consists of normal EEG signals, also referred as interictal signals. FIG. 1 shows recordings of EEG signals from 6 channels during interictal (baseline), preictal (just before a seizure) and ictal (during seizure) period. The goal of seizure detection is to classify parts of the EEG signal as interictal or ictal.

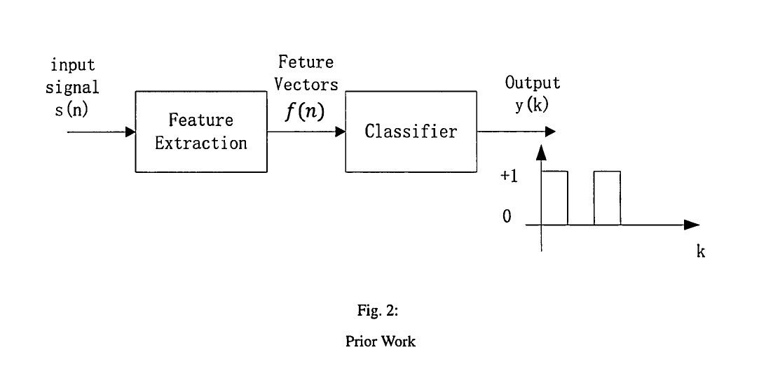

A system architecture for any binary classification is shown in FIG. 2. The seizure detection system also contains 2 parts: (1) feature extraction and (2) classification. Feature extraction step computes discriminant features for the classifier from a single channel EEG signal. If the features are selected properly such that the between-class distance is large and within-class vectors are clustered closely, then the classifier will achieve a high sensitivity and specificity.

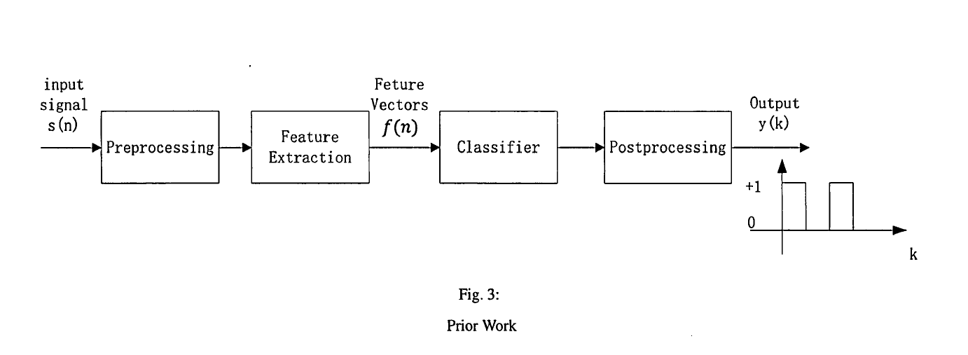

Many seizure detection methods have been proposed based on the system architecture shown in FIG. 2. In order to enhance the detection performance, the systems are modified to include preprocessing the input signal before the features are extracted and post-processing the output of the classifier before the final decision is made. This is described by the system architecture shown in FIG. 3.

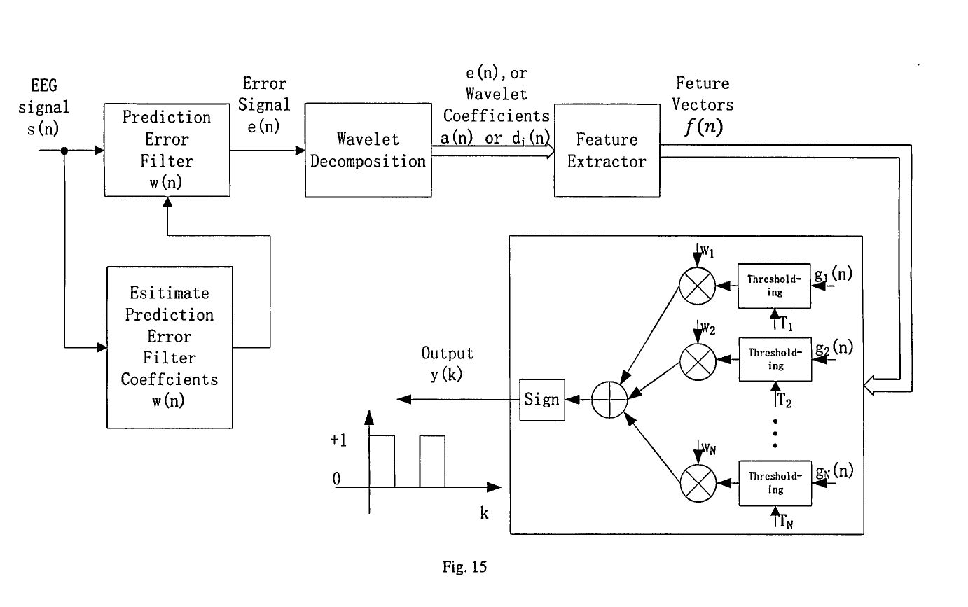

This invention presents a new seizure detection method that requires less hardware complexity and power consumption. FIG. 15 describes a block diagram of this invention comprising 3 parts: (1) Prediction Error Filter (PEF), (2) wavelet feature extraction, and (3) classifier.

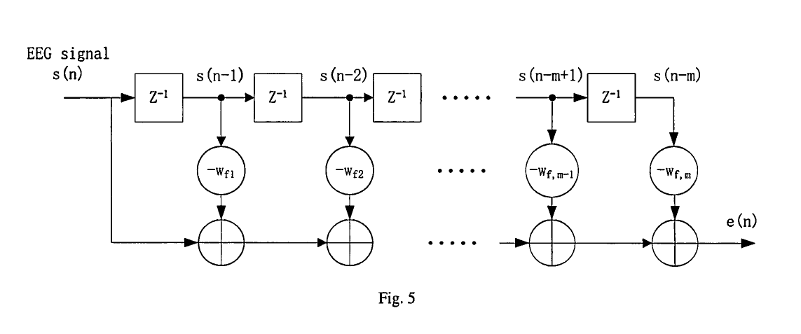

In the first step, EEG data is preprocessed to remove its mean. The demeaned signal is then whitened by using a prediction error filter (PEF). Since EEG data is a non-stationary signal, the input data is divided into several overlapping or non-overlapping segments using a window function. For each segment of data, a PEF is applied to compute the whitened signal e(n). FIG. 5 describes a block diagram of the PEF. The coefficients of this filter are computed by:

w=R⁻¹r (1)

where w describes the coefficients of the PEF,

$$R = [ \begin{matrix}r ( 0 ) & r ( 1 ) & … & r ( M - 2 ) & r ( M - 1 ) \\ r ( 1 ) & r ( 0 ) & … & r ( M - 3 ) & r ( M - 2 ) \\ … & … & … & … & … \\ … & … & … & … & … \\ ⋯ & … & … & … & … \\ r ( M - 2 ) & r ( M - 3 ) & … & r ( 0 ) & r ( 1 ) \\ r ( M - 1 ) & r ( M - 2 ) & … & r ( 1 ) & r ( 0 )\end{matrix} ] \qquad ( 2 )$$

represents the autocorrelation matrix of the input sample vector of a window, and r=[r(1), r(2), . . . , r(M)] represents the cross-correlation vector between the input sample vector and its delayed version. In one embodiment, the filter coefficients can be estimated using data corresponding to a small duration and then be used over long period of time. In another embodiment, the filter coefficients can be estimated more often. The filter coefficients w computed from from Eq. (1) are also often referred as the Wiener filter. The prediction error filter coefficients (w) can be adapted by recomputing the auto-correlation matrix R in Eq. (2) and using this R in Eq. (1) to compute w. In an implantable device, the w coefficients can be uploaded by a radio frequency link. The w coefficients can be programmed in the device at an appropriate frequency.

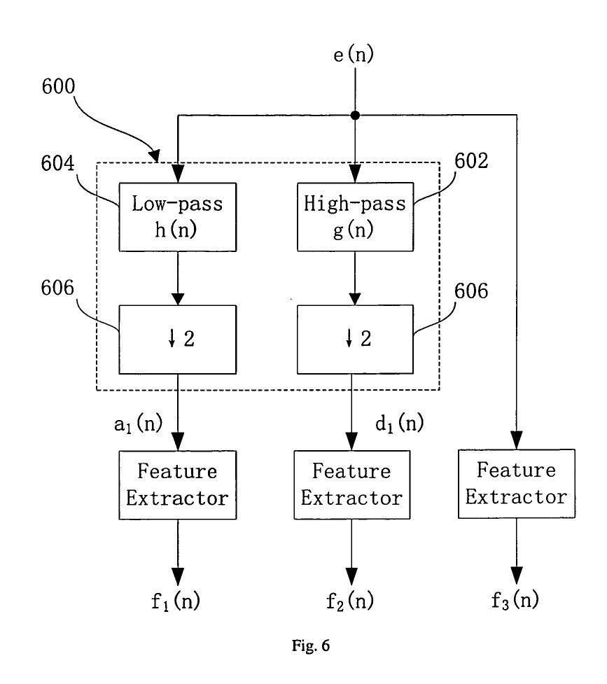

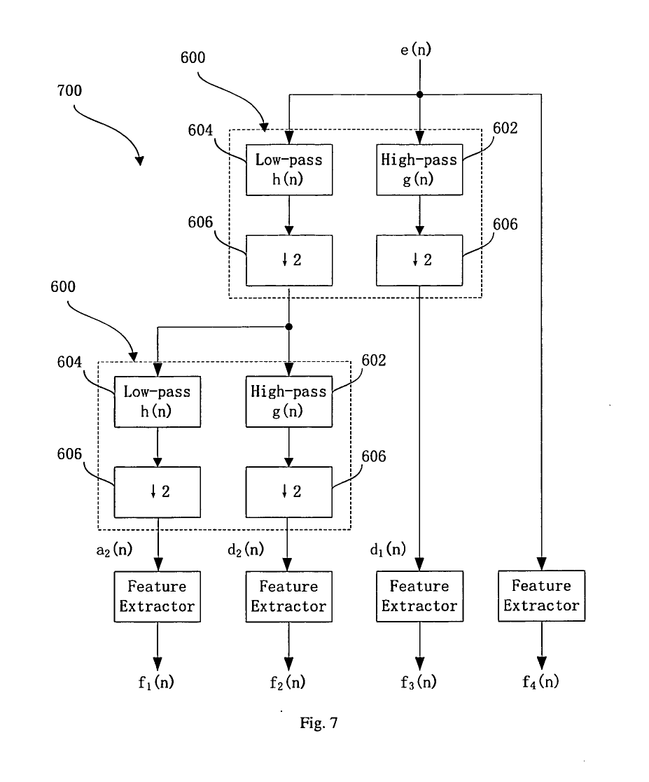

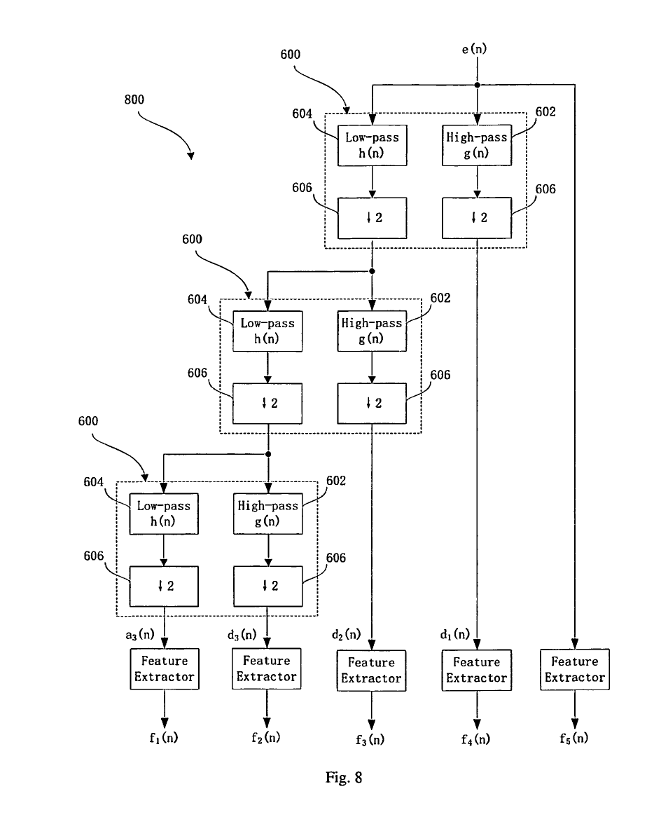

In the second step, wavelet decomposition is applied to the error signal to compute different wavelet coefficients. Several features can then be computed from these wavelet coefficients. The error signal can be considered as 0-level wavelet coefficients. A block diagram of a one-level wavelet decomposition 600 is shown in FIG. 6. Block 600 consists of a high-pass filter 602, a low-pass filter 604 and 2 downsamplers 606 each of which downsamples by a factor of 2. The output a₁(n) and d₁(n) are called first-level approximate coefficients and first-level detail coefficients, respectively. FIG. 6 also shows the features extracted from the error signal and first-level wavelet coefficients. FIG. 7 and FIG. 8 show block diagrams of a 2-level wavelet decomposition and a 3-level wavelet decomposition using 2 and 3 repetitions of the block 600, respectively, where the approximate coefficients of the previous level are further decomposed into approximate and detail coefficients. In various embodiments, the filters h(n) and g(n) can correspond to coefficients from Haar wavelet, symlets, or Daubechies wavelets, etc. FIG. 7 and FIG. 8 also illustrate the extraction of features from the wavelet coefficients. It may be noted that prior work has been based on seizure detection using wavelet coefficients of the EEG signal. This invention differs from prior work in the sense that the wavelet decomposition is applied to the error signal and not to the EEG signal. The wavelet decomposition of the error signal is a key component of this invention.

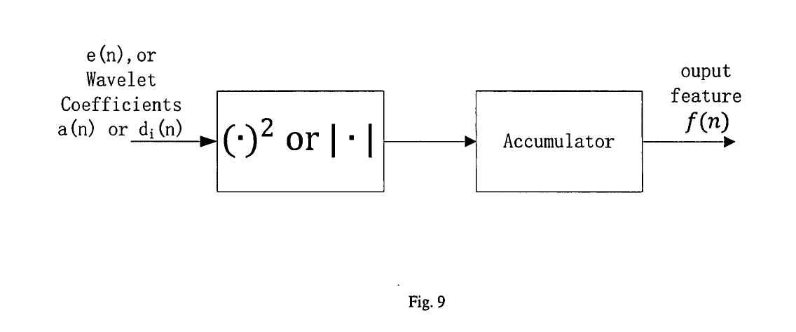

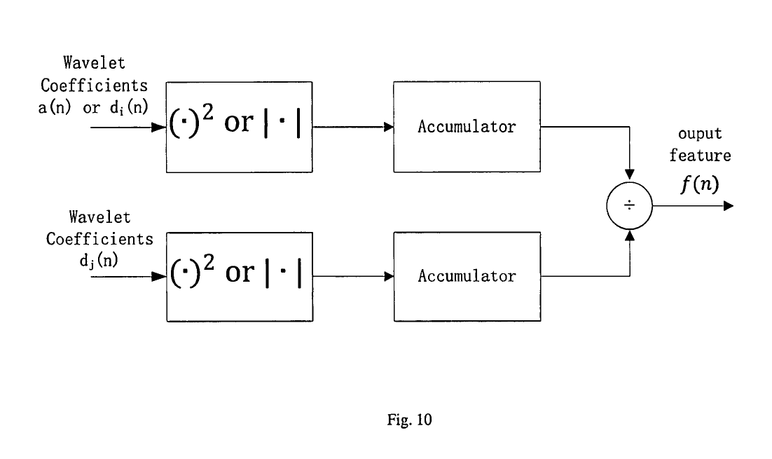

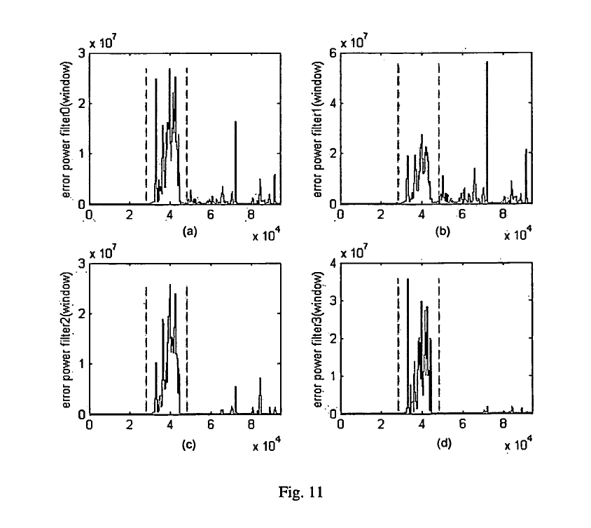

Features are then computed based on the amplitude of the wavelet coefficients of the error signal. In addition, features are also computed from the error signal e(n). In various embodiments, features can be computed as (1) mean squared, (2) mean absolute value or other functions of the amplitude of wavelet coefficients at each level. A block diagram of feature extraction is shown in FIG. 9. In another embodiment, other features can also be computed that correspond to a ratio of the previously said features. FIG. 10 shows a block diagram of such a feature that represents a ratio of power in 2 different bands. We define feature vector at time n as f(n)=[f₁(n), f₂(n), . . . , f_{d}(n)]^{T}, where d denotes the number of features. FIG. 11 shows 4 features extracted using a 2-level wavelet decomposition and mean-squared coefficients, where a seizure is onset during the time period between the 2 vertical dashed lines marked in the figure.

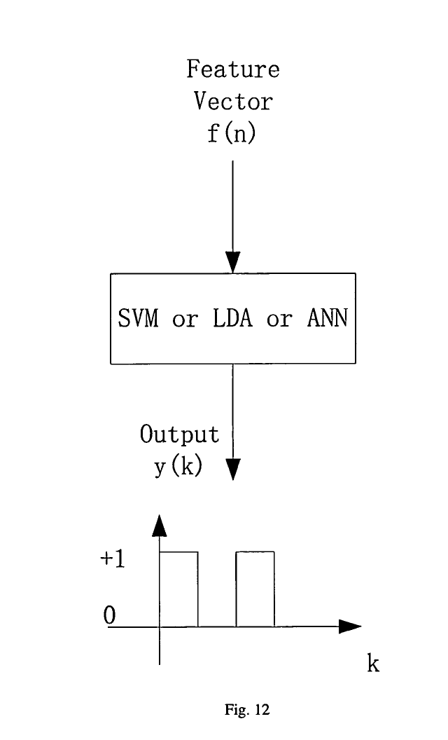

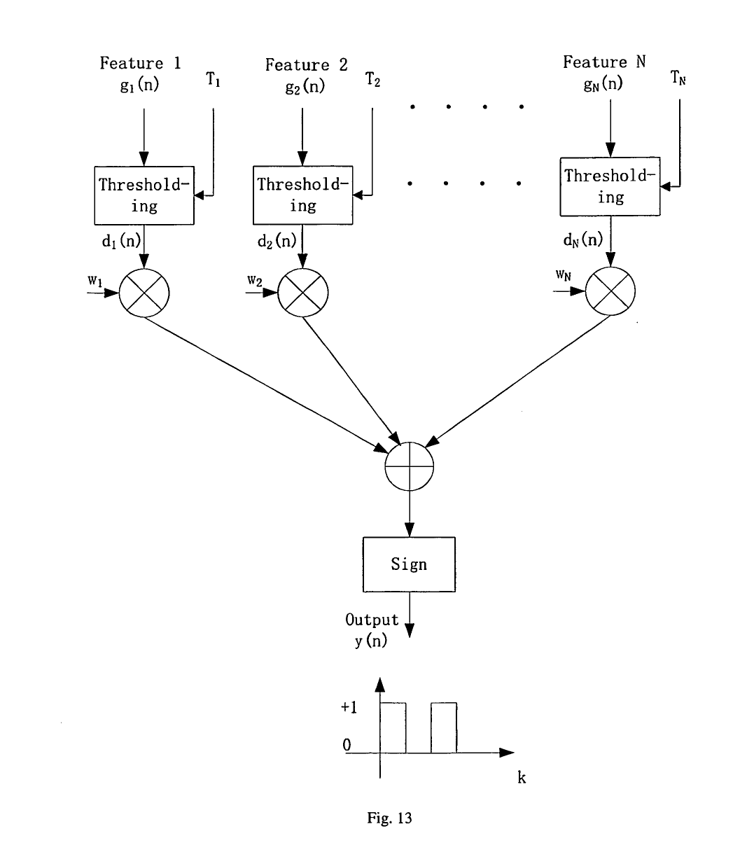

After feature extraction, a classifier is trained to separate feature vectors in ictal period from those in interictal period. A classifier can be a multi-variate classifier. In various embodiments, Support Vector Machine (SVM), Linear Discriminant Analysis (LDA), or Artificial Neural Network (ANN) classifiers can be used. This is illustrated in the block diagram shown in FIG. 12. In one example, linear SVM is used in the classification step. In another example, SVM with radial basis function kernel (RBF-SVM) is used. A classifier can also consist of multiple univariate classifiers trained on a subset of features; these classifiers outputs can then be weighted and summed to compute a final output that is used to generate the final decision. In an embodiment, this said classification method is implemented as an ADABOOST classifier using decision stumps as basic learners. A block diagram of the ADABOOST classifier is shown in FIG. 13. This block diagram shows that N classifiers are combined to compute a decision variable. The features g₁(n), g₂(n), . . . , g_{N}(n) are chosen from the feature set f₁(n), f₂(n), . . . , f_{d}(n). A feature fᵢ(n) can map to one or many gₖ(n) features. The output of the thresholding block is denoted by dᵢ(n) which is defined as:

$${d}_{i} ( n ) = \{ \begin{matrix}- 1 & {g}_{i} ( n ) < {T}_{i} \\ + 1 & {g}_{i } ( n ) \geq {T}_{i}\end{matrix} \qquad ( 3 )$$

where Tᵢ is a threshold parameter. The final output y(n) is given by:

$$y ( n ) = sign ( {\sum }_{i = 1}^{N} {w}_{i} {d}_{i} ( n ) ) \qquad ( 4 )$$

where wᵢ is the weight associated with the i-th classifier and

$$sign ( x ) = \{ \begin{matrix}0 & x < 0 \\ 1 & x \geq 0\end{matrix} \qquad ( 5 )$$

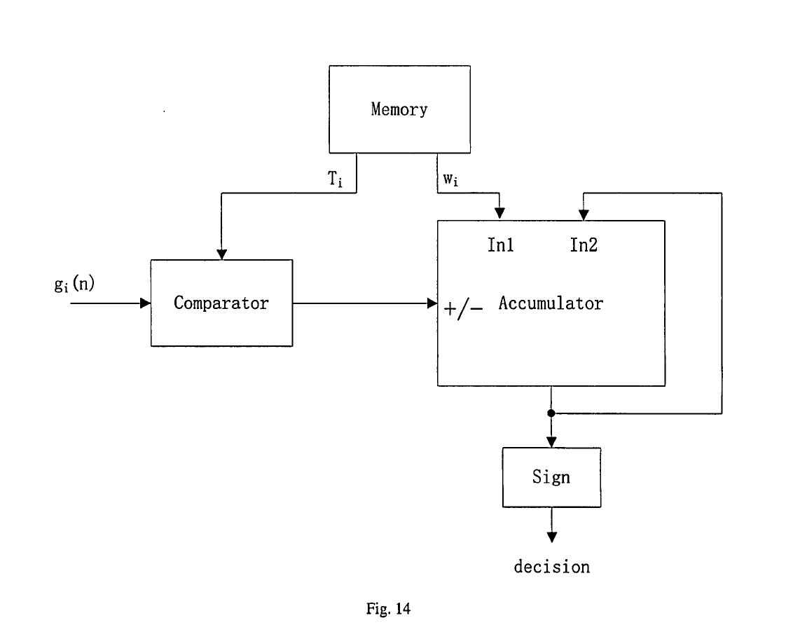

An architecture that implements an ADABOOST classifier using sequential processing approach is shown in FIG. 14. FIG. 15 illustrates a proposed invention of the seizure detection system using ADABOOST classifier.

Once feature vectors are classified, undesired fluctuations can often be encountered. In order to attenuate this phenomenon, which degrades the detection capabilities, it is common to use filtering techniques to smooth such irregular effects. In one embodiment, a Kalman filter is used in the postprocessing step. In other embodiments, a moving-average filter or a median filter can be used in the postprocessing step. In another embodiment, a m-out-of-n selector could be used as the postprocessing step.

The proposed seizure detection algorithm has been tested on the Freiburg database, which is available to public by request. The EEG data in this dataset were obtained using a Neurofile NT digital video EEG system with 128 channels, 256 Hz sampling rate except Patient 12 whose EEG has been sampled at 512 Hz, and a 16-bit analog-to-digital converter.

The Freiburg database contains six contacts of all implanted grid, strip, or depth electrodes: three near the seizure focus (focal) and the other three distal to the focus (afocal).

The database contains electrocorticogram (ECoG) or EEG from 21 patients suffering from medically intractable focal epilepsy. The amount of available data consists of at least 24 hours of interictal recordings for 21 patients with 2-6 seizures and 50 minutes of preictal data. Seizure onset times and artifacts were identified by certified epileptologists.

For each patient, the performance of the proposed system is measured in terms of sensitivity and the false detection rate. Sensitivity, defined as

$$Sensitivity = \frac{\# of TPs}{\# of TPs + \# of FNs} \qquad ( 6 )$$

measures the proportion of the ictal events in a patient that are correctly classified by the proposed algorithm, where TPs represents the true positives and FNs represents the false negatives.

In addition, the false detection rate per hour demonstrates how many false alarms the proposed algorithm would generate in the interictal recordings. An approximately 30-min interval is considered as detection horizon.

The proposed algorithm using SVM classifier with radial basis function (RBF) kernel achieves a high sensitivity of 97.5% and a false detection rate of 0.285 per hour (159 false alarm events in 427.6 interictal hours).

### CONCLUSION

Various embodiments of the present invention can be implemented using different levels of wavelet decomposition, different methods of feature computation and different types of the classifiers. These various embodiments can be implemented in implantable or wearable biomedical devices to trigger a signal when seizures are detected. This trigger signal can be used in the closed-loop therapy system to deliver anti-epileptic drugs or deliver a therapy based on electrical or magnetic stimulation or modulation of the brain. The stimulation could be delivered in an invasive or non-invasive manner.

It should be understood that these embodiments have been presented by way of example only, and not limitation. It will be understood by those skilled in the relevant art that various changes in form and details of the embodiments described may be made without departing from the spirit and scope of the present invention as defined in the claims. Thus, the breadth and scope of present invention should not be limited by any of the above-described exemplary embodiments, but should be defined only in accordance with the following claims and their equivalents.

What is claimed is:

1. A seizure detection system, comprising:
i. a prediction error filter coupled to an EEG signal to compute an error signal;
ii. wavelet decomposition of the error signal to compute wavelet coefficients;
iii. extraction of features from the said wavelet coefficients; and
iv. a classifier to process the said features to detect seizures.

2. The system in claim 1 where the prediction error filter coefficients are fixed.

3. The system in claim 1 where the prediction error filter coefficients are adapted from the EEG signal.

4. The system in claim 1 where a first feature and a second feature are extracted by computing the sums of the squares of the first and second wavelet coefficients.

5. The system in claim 1 where a first feature and a second feature are extracted by computing the sums of the absolute values of the first and second wavelet coefficients.

6. The system in claim 4 where a third feature is extracted by computing the ratio of the first feature and the second feature.

7. The system in claim 5 where a third feature is extracted by computing the ratio of the first feature and the second feature.

8. The system in claim 1 where the classifier is a support vector machine classifier.

9. The system in claim 1 where the classifier is a linear discriminant analysis classifier.

10. The system in claim 1 where the classifier is an ADABOOST classifier.

11. The system in claim 1 implemented by a machine.

12. A seizure detection device, comprising:
i. a digital circuit;
ii. a prediction error filter coupled to an EEG signal to compute an error signal;
iii. wavelet decomposition of the error signal to compute wavelet coefficients;
iv. extraction of features from the said wavelet coefficients; and
v. a classifier to process the said features to detect seizures.

13. The device in claim 12 where the prediction error filter coefficients are fixed.

14. The device in claim 12 to include an adaptation circuit to adapt the prediction error filter coefficients from the EEG signal.

15. The device in claim 12 to include digital circuits to compute a first and a second wavelet coefficients.

16. The device in claim 12 further comprising circuits to compute a first feature and a second feature by computing the sums of the squares of the first and second wavelet coefficients.

17. The device in claim 12 further comprising circuits to compute a first feature and a second feature by computing the sums of the absolute values of the first and second wavelet coefficients.

18. The device in claim 16 to include circuits to compute a third feature by computing the ratio of the first feature and the second feature.

19. The device in claim 17 to include circuits to compute a third feature by computing the ratio of the first feature and the second feature.

20. The device in claim 12 where the classifier implements a support vector machine classifier.

21. The device in claim 12 where the classifier implements a linear discriminant analysis classifier.

22. The device in claim 12 where the classifier implements an Adaboost classifier.

23. The device in claim 12 to create a trigger for therapy delivery.

24. A seizure detection device, comprising:
i. a digital circuit;
ii. a prediction error filter coupled to an EEG signal to compute an error signal;
iii. wavelet decomposition of the error signal to compute wavelet coefficients;
iv. extraction of features from the said wavelet coefficients; and
v. a classifier, further comprising:
a. thresholding a plurality of features to compute a plurality of decisions;
b. computing a weighted sum of these decisions to detect seizures.

25. The device in claim 24 to create a trigger for therapy delivery.
